(19) United States
(12) Patent Application Publication
(10) Pub. No.: US 2023/0118618 A1
(43) Pub. Date: Apr. 20, 2023

(54) METHOD AND APPARATUS FOR DELIVERY OF ACTIVE PHARMACEUTICAL INGREDIENTS INTO SMOKABLE STRUCTURES

(71) Applicant: Transport Authority, Inc., Dover, DE (US)

(72) Inventors: William Warren, Lafayette, CA (US); Walter Lee Smith, Danville, CA (US); David A. Doyle, Sonora, CA (US)

(21) Appl. No.: 18/047,517

(22) Filed: Oct. 18, 2022

Related U.S. Application Data
(60) Provisional application No. 63/256,815, filed on Oct. 18, 2021.

(51) Int. Cl. A24D 1/18; A61M 11/06; A61K 31/352

(52) U.S. Cl. CPC A24D 1/18; A61M 11/06; A61K 31/352

(57) ABSTRACT

Methods and apparatus are described for delivering API to a smokable structures and tea bags that contain a fill material. The fill material can include cannabis plant matter, such as cannabis flowers. In one example, an injection tube can be inserted into a smokable structure, and the API can be emitted as a spray. Other methods examples of delivering API to smokable structures are disclosed.

### CROSS-REFERENCE TO RELATED APPLICATIONS

This claims priority to U.S. Patent Application Ser. No. 63/256,815 filed Oct. 18, 2021, the disclosure of which is hereby incorporated by reference as if set forth in its entirety herein.

### BACKGROUND

Smokable structures generally include a paper or other flammable wrapping material that contains a fill material. The wrapping material is typically formed into a substantially cylindrical or conical shape. Certain smokable structures can be similar to a cigarette in size and shape, but the fill material includes cannabis plant matter, such as ground cannabis flowers, rather than tobacco and nicotine-based chemicals. Such smokable structures are ignited and smoked by consumers to obtain therapeutic or recreational benefits from cannabis active pharmaceutical ingredients (API) including cannabidiol (CBD), tetrahydrocannabinol (THC) and terpenoids. Smokable structures can also include hemp and CBD cigarettes, blunts, and cigars. Similarly, CBD teabags are used for their calming effect and focus improvement. Together, the use of these products is widespread today, exceeding 10 million units per year.

Smokable structures can be provided as a roll or a pre-roll. Pre-roll smokable structures are provided to the user in their constructed form, including the wrapping material that contains the cannabis plant matter. Rolls can be created in situ by individually purchasing the wrapping material and fill material, and subsequently either forming the wrapping material about the fill material, or by first forming the wrapping material and subsequently inserting the fill material.

The potency and quantity of fill material of smokable structures and tea bags can vary widely. Therefore, it can be desirable to provide a method and apparatus for introducing APIs into smokable structures and/or components of smokable structures, including the wrapping and/or the fill material. It can be further desirable to provide a method and apparatus for introducing APIs into tea bags or other media that can create an infusion when exposed to water.

### SUMMARY

In accordance with certain aspects of the present disclosure, methods and apparatus are provided for delivering an active pharmaceutical ingredient to a smokable roll having a fill that includes cannabis, a smokable pre-roll that includes cannabis, or a teabag that includes cannabis.

### BRIEF DESCRIPTION OF THE DRAWINGS

FIG. 1A is a schematic view of a dosing system for delivering an API to a smokable pre-roll having a substantially frustoconical shape, including a reservoir and an injection tube that receives the API from the reservoir and delivers the API to the smokable pre-roll;

FIG. 1B is an enlarged view of a portion of the injection tube;

FIG. 1C is a schematic elevation view of the injection tube inserted into the smokable pre-roll at a first withdrawal position;

FIG. 1D is a schematic elevation view of the injection tube at a second withdrawal position;

FIG. 1E is a schematic elevation view similar to FIG. 1C, but showing the smokable pre-roll in a substantially cylindrical shape;

FIG. 2 is a schematic elevation view of a portion of a dosing system illustrated in FIG. 1A, but including a plurality of injection tubes, wherein the injection tubes are inserted at the same insertion depth;

FIG. 3 is a schematic elevation view of a portion of a dosing system illustrated in FIG. 2, but showing the injection tubes inserted at different insertion depths;

FIG. 4A is a schematic view of another dosing system for delivering an API to a smokable pre-roll, including a reservoir and a dosing head to configured to receive API from the reservoir, and deliver the API to the smokable pre-roll as the fill material is inserted into the outer wrapping;

FIG. 4B is a schematic view of another dosing system for delivering an API to a smokable pre-roll, including a reservoir and a dosing head to configured to receive API from the reservoir, and deliver the API to the smokable pre-roll prior to insertion of the fill material into the outer wrapping in another example;

FIG. 5A is a schematic view of a dosing station configured to deliver API to fill material prior to inserting the fill material into the outer wrapping;

FIG. 5B is a schematic view showing insertion of the fill material of FIG. 5A into the outer wrapping;

FIG. 5C is a schematic view showing insertion of the fill material of FIG. 5A into the outer wrapping in another example;

FIG. 6 is a schematic view of a dosing station configured to deliver API to an outer wrapping of a smokable structure; and

FIG. 7 is a schematic view of a dosing station configured to deliver API to a teabag in another example.

### DETAILED DESCRIPTION

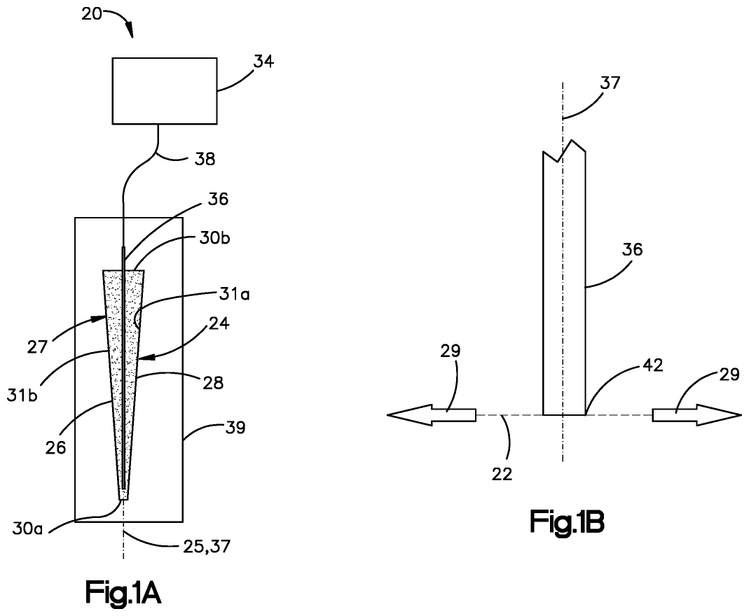

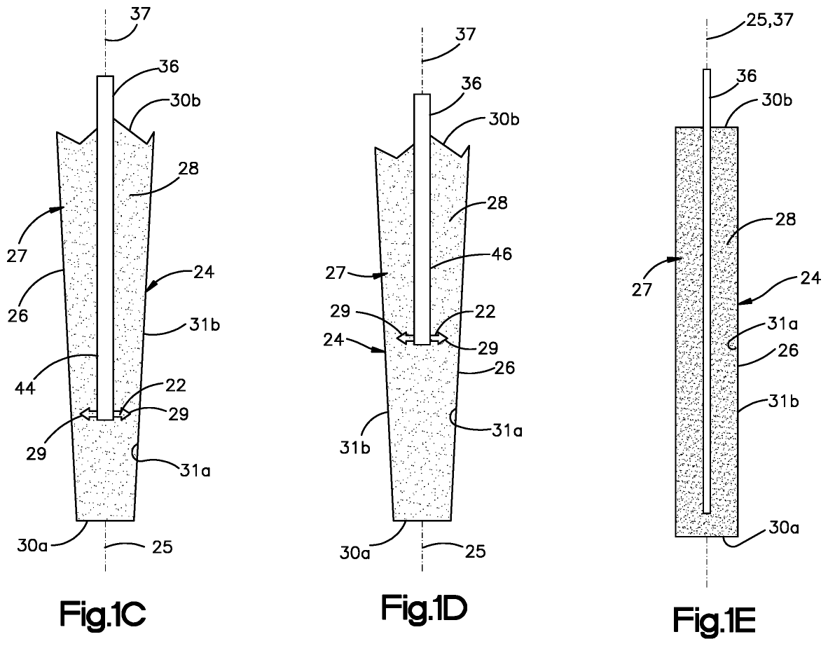

Referring initially to FIG. 1A-1B, a dosing system 20 can be configured to deliver an API 22 to a pre-roll smokable structure 24, also referred to herein as a pre-roll. The pre-roll 24 can include an outer wrapping 26 and fill material 28 disposed in the outer wrapping 26, such that the outer wrapping 26 surrounds the fill material 28. In some examples, the outer wrapping 26 is formed to define an internal space 27 and the fill material 28 is inserted into the internal space 27. In particular, the outer wrapping 26 can define an inner surface 31a that defines the internal space 27, and an outer surface 31b that is radially opposite the inner surface 31a. In other examples, the fill material 28 is placed on the outer wrapping 26, and the outer wrapping is subsequently formed about the fill material 28, such that the fill material 28 is disposed in the internal space 27.

The pre-roll 24 can be elongate along a central axis 25, and can define a first open end 30a and a second open end 30b opposite the first end 30a along the central axis 25. Each of the first and second open ends 30a and 30b can be open to the internal space 27. In one example, the fill material 28 can include combustible cannabis plant material, such as cannabis flower. Prior to surrounding the fill material 28 with the outer wrapping 26, the fill material 28 can be dried as desired so as to remove water molecules that could otherwise impeded diffusion of the API into the internal space 27 for absorption by the fill material 28. The outer wrapping 26 can be made from smoking paper, tobacco, or any suitable combustible material that is configured to burn in a controlled manner during combustion of the fill material 28. The first end 30a can define a draw end, and the second end 30b can define a tip that is configured to be ignited. The second end 30b is ignited, while the user inhales from the first end 30, thereby ingesting the smoke created as the fill material 28 burns. Thus, user can thus experience the therapeutic effects of the fill material 28. The pre-roll can be configured as a cigarette, a blunt, a cigar, or the like.

The outer wrapping 26 can define any suitable size and shape as desired. For instance, in one example, the outer wrapping 26 can be substantially frustoconical in shape about the central axis 25. The first and second ends 30a and 30b define respective cross-sectional areas along a direction perpendicular to the central axis 25. When the outer wrapping 26 is frustoconical, the cross-sectional area of the second end 30b is greater than the cross-sectional area of the first end 30a. Alternatively, as shown at FIG. 1E, the outer wrapping 26 can be substantially cylindrical in shape about the central axis 25, such that the cross-sectional dimensions of the first and second ends 30a and 30b are substantially equal to each other. The term “substantially” with respect to the shape of the outer wrappings 26 recognizes that the actual shape is determined, at least in part, by the geometry defined by the fill material 28, which can cause the actual shape of the outer wrapping 26 to deviate from the geometric shapes such as the frustoconical and cylindrical geometric shapes.

Referring again to FIGS. 1A-1B, the present inventors have recognized that it can be desirable to deliver at least one API 22 to the pre-roll 24, such that the at least one API 22 can also be inhaled as the pre-roll 24 is burned. The at least one API can include at least one of a cannabinoid, such as cannabidiol or tetrahydrocannabinol, and one or more terpenoids. For instance, the cannabinoid or terpenoid can be dissolved in a carrier fluid to create a liquid API. Therefore, the dosing system 20 can be configured to deliver the API 22 to the pre-roll 24. The API 22 can enhance the therapeutic effect of smoking the pre-roll 24. Alternatively or additionally, the one or more terpenoids can provide a desired fragrance of the pre-roll as it is smoked. As will be appreciated from the description below, the dosing system 20 can repeatably deliver the API 22 to the pre-roll 24 at predetermined locations in the fill material 28, both along the central axis 25 and along directions perpendicular to the central axis 25. In one example, the dosing system 20 can include a reservoir 34 that can contain the API 22 in liquid form, an injection tube 36, and a conduit 38 that extends from the reservoir 34 to the injection tube 36. Thus, the API 22 can travel from the reservoir 34 through the conduit 38 to the injection tube 36. The dosing system 20 can further include one or more pumps, valves, and the like as desired that cause the API 22 to be delivered from the reservoir 34 to the injection tube 36.

The injection tube 36 can be configured as a conventional needle, or other structure suitable to deliver API to the interior space 27 as desired. The injection tube 36 can define an inlet end that receives the API 22 from the conduit 38, and an outlet end that is opposite the inlet end and configured to dispense the API 22. The injection tube 36 can be elongate along a respective central tube axis 37. The injection tube 36 can be cannulated from the inlet end to the outlet end. The injection tube 36 can define an injection orifice 42 at the outlet end. It is recognized that the cross-sectional outlet area of the injection orifice 42 can be either predetermined or adjustable to emit a desired volume of API 22 correlated to a desired pressure of API 22 in the injection tube 36. In this regard, the volume of API 22 that travels out of the injection orifice 42 can be tuned to a rate of infusion of the API 22 in the internal space 27 and absorption of the API 22 by the fill material 28.

During operation, the dosing system 20 can deliver the API to the pre-roll 24. In particular, the injection tube 36 can be inserted into the fill material 28, such that the outlet end is surrounded by the fill material 28. In particular, the injection tube 36 can be inserted into the fill material 28 along a direction that is defined by the central axis 25. For instance, the injection tube 36 can be inserted into the fill material 28 substantially along the central axis 25. The injection tube 36 can be inserted into the second end 30b along a direction toward the first end 30a to a position whereby the exit orifice 42 is disposed adjacent the first end 30a. In particular, the exit orifice 42 can be disposed close enough to the first end 30a such that the API delivered from the exit orifice 42 can be burned during use, but not so close such that a substantial quantity the API delivered from the exit orifice 42 travels out the first end 30a.

Once the injection tube 36 has been inserted into the fill material 28, the API 22 can be driven through the injection tube 36 and out the exit orifice 42. Thus, the API 22 can be delivered from the exit orifice 42 and into the fill material 28. In one example, some or all of the API 22 can be expelled from the exit orifice 42 along a direction that is defined by the central tube axis 37. Alternatively or additionally, some or all of the API can be expelled from the exit orifice 42 along a direction that is angularly offset, such as perpendicular, with respect to the central tube axis 37. Thus, at least a portion of the API can be expelled from the exit orifice 42 radially along a direction toward the outer wrapping 26 as indicated by arrows 29. For instance, the exit orifice 42 can be defined by a nozzle that delivers the API along multiple directions that are angularly offset with respect to the central axis 25. Thus, a quantity of the API can be delivered to a location that is beyond midway from the central axis to the outer wrapping. For instance, in some examples at least 25% of the API that is delivered from the injection tube 36 can travel to a location that is beyond midway from the central axis to the outer wrapping. For instance, the API can travel to the outer wrapping. In one example, the API 22 can be delivered radially 360 degrees about the central axis 25.

In one example, and in all examples and embodiments described herein unless indicated to the contrary, the API can be delivered as successive microdroplets described in U.S. Publication No. 2020/0352826 published on Nov. 12, 2020, included herein as Appendix A which forms part of the present disclosure. Alternatively, the nozzle that defines the exit orifice 42 can be configured as a spray nozzle, such that the API 22 is delivered from the spray nozzle as an atomized spray. Alternatively still, the API 22 can exit as a stream. Alternatively, the API 22 can exit in the form of conventional droplets or any other form as desired.

As described above, and as shown at FIG. 1A, the injection tube 36 can be injected in a distal direction to an insertion position whereby the exit orifice 42 is disposed proximate to the first open end 30a. The distal direction can be defined as a direction from the second open end 30b toward the first open end 30a. In one example, the API can be driven out the exit orifice 42 as the injection tube 36 is withdrawn in a proximal direction that is opposite the distal direction. The proximal direction can thus be referred to as a withdrawal direction, and the distal direction can be referred to as an insertion direction. Thus, the proximal direction can be defined as a direction from the first open end 30a toward the second open end 30b. Thus, the API 22 can be delivered to the fill material 28 either intermittently or continuously along the length of the pre-roll 24.

For instance, as illustrated in FIGS. 1C-1D, the injection tube 36 is shown at a first withdrawal position 44 that is proximal of the fully inserted position of FIG. 1A, and a second withdrawal position 46 that is proximal of the first withdrawal position 44. Of course, the injection tube 36 can define any number of withdrawal positions as desired. In one example, the injection tube 36 is withdrawn from the fully inserted position shown in FIG. 1, and pauses at each of the first and second withdrawal positions 44 and 46. In one example, the API 22 is selectively delivered from the exit orifice 42 when the injection tube 36 is in the fully inserted position of FIG. 1A, and at each of the withdrawal positions. In this example, the API 22 is not delivered between withdrawal positions. Thus, the API 22 can be delivered to predetermined locations of the pre-roll 24. In one example, the predetermined locations can be spaced from each other a sufficient distance along a direction parallel to the central axis 25 such that the internal space 27 defines discrete zones of dosed API 22 that are spaced from each other. The discrete zones of dosed API 22 can increase the surface area of API 22 in the internal space 27. Alternatively, adjacent zones of the plurality of discrete zones of API 22 can be adjoined at necks of API 22. In one example, the necks have a cross-sectional dimension no greater than one-half the overall cross-sectional dimension of each of the zones of API 22 that are adjoined by the necks along a direction parallel to the cross-section of the respective necks.

In another example, the API 22 can be delivered as the injection tube 36 travels proximally from the fully inserted position. Thus, the API 22 can be delivered continuously along the length of the injection tube 36. Alternatively, the API 22 can be expelled from the exit orifice 42 in predetermined pulses such that the API 22 is expelled from the injection tube 46 intermittently, whereby the API 22 delivered in response to each pulse can define a discrete zone of API 22 as described above. Thus, successive pulses of API delivery can define adjacent discrete zones of API 22. In still other examples, the API 22 can be delivered as the injection tube 36 travels distally from the second end 30b toward the first end 30a, either at discrete locations, continuously as the injection tube 36 travels distally, or intermittently as the injection tube 36 travels distally.

It is recognized that substantially equal quantities of API 22 can be delivered along the length of the pre-roll 24. Alternatively, the quantity of API 22 can increase in the distal direction. Alternatively still, the quantity of API 22 can increase in the proximal direction. In other examples, for instance when the pre-roll 24 is substantially frustoconical in shape, the quantity of API 22 can increase in the proximal direction but the ratio of the volume of API 22 per volume of fill material can be substantially constant. In still other examples, the ratio of the volume of API 22 per volume of fill material can increase in the proximal direction. Alternatively, ratio of the volume of API 22 per volume of fill material can decrease in the proximal direction.

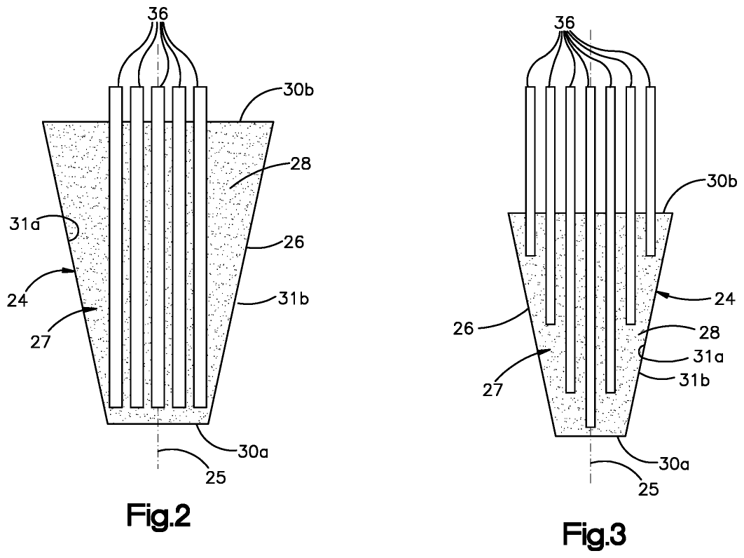

As shown in FIGS. 1A-1E, a single injection tube 36 can be inserted into the pre-roll for the delivery of API to the fill 28. Alternatively, as shown at FIGS. 2-3, the dosing system 20 can include a plurality of injection tubes 36. The injection tubes 36 can receive the API 22 from the same reservoir, or can receive the API from different respective reservoirs. In one example, the API 22 delivered from the injection tubes 36 is the same API. In other examples, the injection tubes 36 can deliver different APIs. The injection tubes 36 can be spaced from each other along a direction that is perpendicular to the central axis 25. The injection tubes 36 can be equidistantly spaced or variably spaced along the direction that is perpendicular to the central axis 25. Thus, the API delivered by the injection tubes 36 is spaced from each other along a direction that is perpendicular to the central axis 25.

In this regard, the location and quantity of the injection tubes 36 can determined the location of API that is dosed in the fill material 28. For instance, the dosing system 20 can be configured to deliver API in a cross-sectional direction that is perpendicular to the central axis 25. Accordingly, the API can be more homogenous along the cross-sectional direction as compared to conventional API dosing systems. The injection tubes 36 can be oriented substantially parallel to each other as they are inserted into the fill material 28. As shown in FIG. 2, the exit orifices 42 of the injection tubes can be substantially aligned with each other along a plane that is oriented perpendicular to the central axis 25. The exit orifice of each of the injection tubes 36 can remain aligned with each other along a plane as active pharmaceutical ingredient is delivered from the exit orifices 42 in the manner described above. Alternatively, the exit orifice of at least one of the injection tubes is inserted to a different depth with respect to the exit orifice of at least one other of the injection tubes as shown in FIG. 3. The injection tubes 36 of FIGS. 2 and 3 can be withdrawn at the same rates or at different rates, and the API 22 can be dispensed as the injection tubes 36 are withdrawn in the manner described above.

Referring again to FIG. 1A, whether the dosing system 20 includes a single injection tube 36 or a plurality of injection tubes 36, the pre-roll 24 can be disposed in a chamber 39 having internal temperature control. Further, the injection tube 36 can be made from a thermally conductive material, such as metal, and can be coupled to a heat source. The heat source can be activated so as to deliver heat to the injection tube 36, which thereby also heats the API 22 that travels through and out the injection tube 36. The API 22 can therefore be heated prior to travelling out of the injection tube 36 so as to decrease the viscosity of the API as it is delivered to the internal space. The decreased viscosity can increase the flowability of the API 22 once it has travelled out of the dosing head 52. Thus, the API 22 can travel radially, for instance, a greater distance than API 22 that has not been heated. Once it has been determined that the API 22 has reached or has approached a desired location in the internal space 27, the temperature of the chamber 39 can be subsequently rapidly cooled to a temperature that is less than the ambient temperature and sufficient to quench the API 22, thereby preventing significant further travel of the API in the internal space 27. Further, the subsequent cooling can prevent or limit decarboxylation of the API 22. The temperature of the chamber 39 can be subsequently cooled to a temperature that is less than the ambient temperature and sufficient to quench the API 22. Thus, the API 22 can flow from the injection tube 36 to a desired location, and the temperature of the chamber 39 can be subsequently rapidly cooled, thereby quenching the API 22 and preventing significant further travel of the API in the interior space 27. Further, the subsequent cooling of the API 22 can prevent or limit decarboxylation of the API 22.

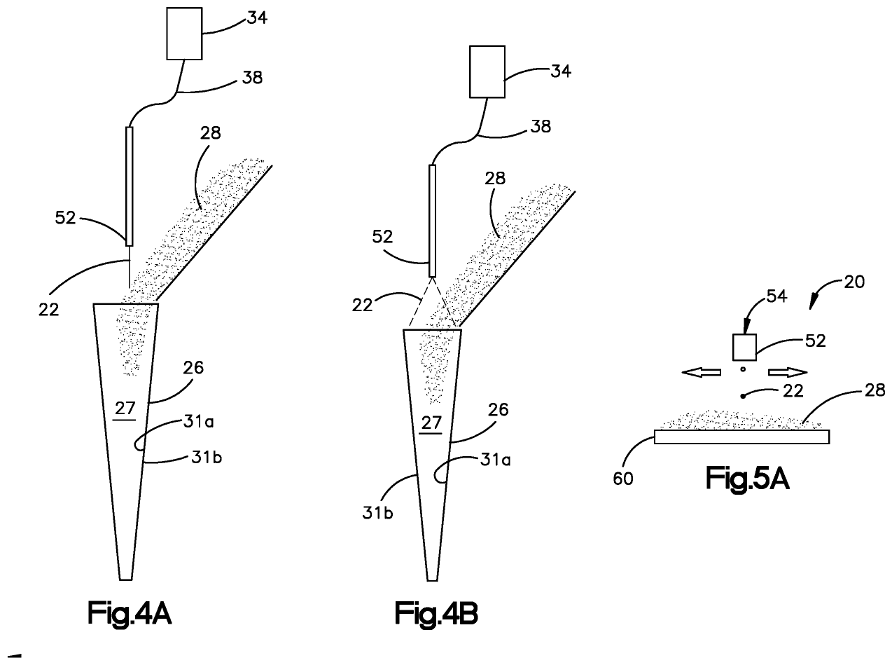

Referring now to FIG. 4A, the dosing system 20 can be configured to deliver the API 22 to the smokable pre-roll 24 as the fill material 28 is inserted into the outer wrapping 26. In particular, the outer wrapping 26 can be formed into a respective shape so as to define the internal space 27. The outer wrapping can define a substantially frustoconical shape, a substantially cylindrical shape, or any suitable alternative shape as desired. The dosing system 20 can include the components as described above, but can include a dosing head 52 that delivers the API 22 into the internal space 27 as the fill material 28 delivered into the internal space 27. In particular, the API can be delivered to either or both of the fill material and the outer wrapping 26 as the fill material is inserted into the internal space 27.

The dosing head 52 can be configured to deliver the API 22 as a spray, as one or more streams, as conventional droplets, or as microdroplets or any other form as desired. The dosing head 52 can be configured as described in Appendix A in some examples. The dosing head 52 can be disposed above the second end 30b, and in particular spaced from the second end 30b along the proximal direction. As the API 22 is dispensed by the dosing head 52, the API 22 is brought into contact with either or both of the fill material 28 as it is inserted into the internal space 27 and the outer wrapping 26. In particular, the API can be brought into contact with the inner surface 31a. When the fill material 28 is disposed in the internal space, and the pre-roll has been fully constructed, the API 22 can be disposed in the internal space 27 along both the length of the pre-roll 24 and along cross-sections of the pre-roll that are perpendicular to the central axis 25.

As described above with respect to FIG. 1A, the pre-roll 24 can be disposed in the chamber 39 having internal temperature control. Further, as described above, the injection tube 36 can be made from a thermally conductive material, such as metal, and can be coupled to a heat source. The heat source can be activated so as to deliver heat to the injection tube 36, which thereby also heats the API 22 that travels through and out the injection tube 36. The API 22 can therefore be heated prior to travelling out of the dosing head 52 so as to decrease the viscosity of the API as it is delivered to the internal space. The decreased viscosity can increase the flowability of the API 22 once it has travelled out of the dosing head 52. The temperature of the chamber 39 can be subsequently rapidly cooled to quench the API 22 in the manner described above. Thus, the heated API 22 can flow from the injection tube 36 to a desired location in the internal space 27, and the temperature of the chamber 39 can be subsequently rapidly cooled to quench the API 22 and preventing significant further travel of the API in the interior space 27. Further, the subsequent cooling can prevent or limit decarboxylation of the API 22.

Referring now to FIG. 4B, the dosing system 20 can be configured to deliver the API 22 to the pre-roll 24 as the fill material 28 is inserted into the outer wrapping 26 in another example. In particular, the outer wrapping 26 can be formed in the manner described above, and the API 22 can be delivered from the dosing head 52 to the inner surface 31a of the outer wrapping 26. The outer wrapping 26 can be oriented such that the second outer end 30b is disposed above the first outer end 30a. In one example, the central axis 25 can be oriented vertically. Thus, the API 22 can be delivered from the dosing head 52 to the inner surface 31a, such that the API 22 can flow in the distal direction toward the first end 30a under gravitational forces, thereby coating at least a portion of the inner surface 31a. The API 22 can be delivered from the dosing head 52 at the second end 30b, such that the API 22 travels toward the first end. In some examples, it may also be desired to also deliver the API 22 from the dosing head 52 to the inner surface 31a at one or more locations between the second end 30b and the first end 30a. The API 22 can be delivered as a spray in some examples so as to coat the inner surface 31a about the central axis 25. Alternatively, the API 22 can be delivered as a stream, as microdroplets, as conventional droplets, or in any form as desired. In this regard, a single dosing head 52 can deliver the API 22 to the inner surface 31a, or multiple dosing heads 52 can deliver 52 that API 22 to different respective locations of the inner surface 31a. In this manner, the API 22 can be disposed in the pre-roll 24 after the fill material 28 has been inserted into the internal space 27. The API 22 can be heated as it travels out of the dosing head 52 and subsequently quenched in the manner described above, so as to control the distance that the API 22 flows 22 along the inner surface 31a.

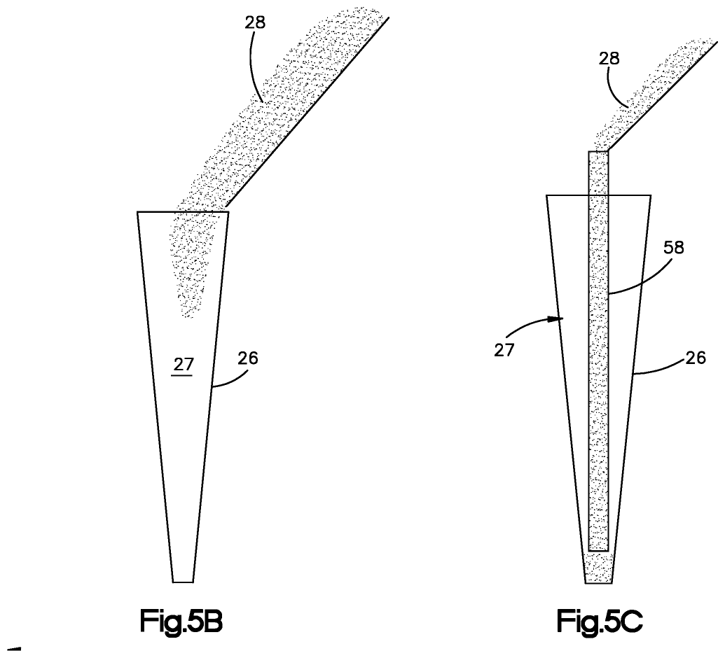

Referring now to FIG. 5A, the dosing system 20 can be configured as a dosing station 54 that is configured to deliver the API 22 to the fill material 28 in bulk, prior to surrounding the fill material 28 with the outer wrapping 26. The dosing station 54 can be configured as described in Appendix A, and can thus be configured to deliver the API 22 from at least one dosing head 52 to the fill material 28. In particular, the fill material 28 can be brought into alignment with the dosing head 52 by moving the fill material 28 into alignment with the dosing head 52 and/or moving the dosing head 52 into alignment with the fill material 28. In one example, the fill material 28 can be disposed on a conveyor 60 that delivers the fill material 28 to a position in alignment with the at least one dosing head 52. The API 22 can then be delivered from the dosing head 52 to the fill material 28.

In one example, the API 22 can be delivered to the fill material 28 under gravitational forces. For instance, the API 22 can be delivered as successive microdroplets. Alternatively, the API can be delivered as a stream, as a spray, as conventional droplets, or in any suitable alternative form as desired. The fill material 28 can then be surrounded by the outer wrapping 26. In particular, as described above, the fill material 28 can be placed onto the inner surface 31a of the outer wrapping 26, and the outer wrapping 26 can then be formed into its geometric shape about the fill material 28 so as to define the internal space 27. Thus, the outer wrapping 26 surrounds the fill material 28. Alternatively, the outer wrapping 26 can be formed into its geometric shape so as to define the internal space 27, and the fill material 28 can subsequently be inserted into the internal space 27 as illustrated in FIG. 5B. If the fill material 28 dosed with the API 22 is too tacky or sticky for easy insertion into the internal space 27, a guide member 58 having a smooth surface can be inserted into the internal space 27, and the fill material 28 can be inserted along the guide member 58 into the internal space 27, as shown in FIG. 5C.

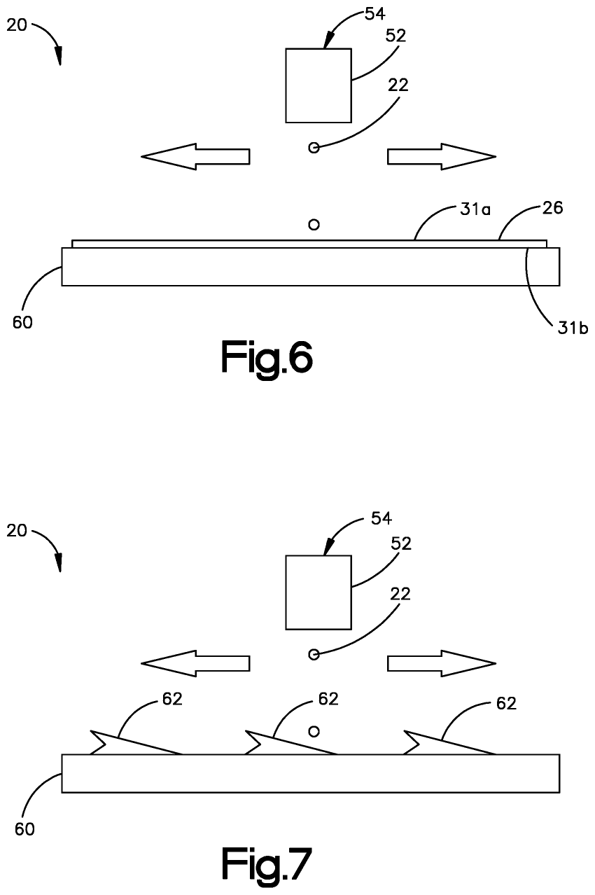

Referring now to FIG. 6, the dosing system 20 can be configured as a dosing station 54 that is configured to deliver the API 22 to at least one outer wrapping 26 prior to surrounding the fill material 28 with the outer wrapping 26. In one example, the at least one outer wrapping 26 can be provided as a sheet that defines a plurality of outer wrappings 26 can be subsequently cut into a plurality of outer wrappings 26, or can be delivered to individualized outer wrappings 26 as desired. The at least one outer wrapping 26 can be substantially planar as the API 22 is delivered to the outer wrapping. The dosing station 54 can be configured as described in Appendix A, and can thus be configured to deliver the API 22 from at least one dosing head 52 to the outer wrapping 26. In particular, the outer wrapping 26 can be brought into alignment with the dosing head 52 by moving the outer wrapping 26 into alignment with the dosing head 52 and/or moving the dosing head 52 into alignment with the outer wrapping 26. In one example, the outer wrapping 26 can be disposed on a conveyor 60 that delivers the outer wrapping 26 to a position in alignment with the at least one dosing head 52. The API 22 can then be delivered from the dosing head 52 to the outer wrapping 26. In one example, the API 22 can be delivered to the inner surface 31a of the outer wrapping 26. Alternatively, the API 22 can be delivered to the outer surface 31b of the outer wrapping 26. Alternatively still, the API 22 can be delivered to one of the inner and outer surfaces 31a-b, and the outer wrapping 26 can then be flipped so that the API 22 can be delivered to the other of the inner and outer surfaces 31a-b.

In one example, the API 22 can be delivered to the outer wrapping 26 under gravitational forces. For instance, the API 22 can be delivered as successive microdroplets. Alternatively, the API 22 can be delivered as a stream, as a spray, as conventional droplets, or in any suitable alternative form as desired. The outer wrapping 26 can then surround the fill material 28. In particular, as described above, the fill material 28 can be placed onto the inner surface 31a of the outer wrapping 26, and the outer wrapping 26 can then be formed into its geometric shape about the fill material 28 so as to define the internal space 27. Thus, the outer wrapping 26 surrounds the fill material 28. Alternatively, the outer wrapping 26 can be formed into its geometric shape so as to define the internal space 27, and the fill material 28 can subsequently be inserted into the internal space 27. Alternatively, the outer wrapping 26 can be sold to the consumer who can then form his or her smokable roll in situ.

Referring now to FIG. 7, the methods and apparatus described above can further be applied to deliver the API 22 to a teabag 62. The teabag 62 includes an outer liquid-permeable medium that can define an encasement, and a fill material disposed in the encasement. The teabag 62 can be placed in water, such that active ingredients of the fill material is transferred into the water. The water can then be consumed to ingest the active ingredients. In some examples, the fill material can include cannabis plant material, such as cannabis flower. The API 22 can be added to the teabag 62 in any manner as described above. For instance, as illustrated in FIG. 7, the teabag 62 can be brought into alignment with one or more dosing heads 52 to deliver the API 22 to the teabag 62. For instance, the API 22 can be delivered to the outer surface of the liquid-permeable medium. Alternatively, the API 22 can be delivered to the fill material prior to surrounding the fill material with the liquid-permeable medium. Alternatively still, the API 22 can be delivered to the liquid-permeable medium prior to surrounding the fill material with the liquid-permeable medium.

In should be noted that the illustrations and discussions of the embodiments and examples shown in the figures are for exemplary purposes only and should not be construed as limiting the disclosure. One skilled in the art will appreciate that the present disclosure contemplates a range of possible modifications of the various aspect, embodiments, and examples described herein. Additionally, it should be understood that the concepts described herein may be employed along or in combination with any of the other embodiments and examples described herein. It should be further appreciated that the various alternatives described above with respect to one illustrated embodiment can apply to all other embodiments and examples described herein, unless otherwise indicated. Reference is therefore made to the claims.

We claim:

1. A method for delivering a liquid active pharmaceutical ingredient to a pre-roll smokable structure that includes an outer wrapping that is elongate substantially along a central axis, and a fill material disposed in the outer wrapping, the fill material including a cannabis, the method comprising the steps of:
inserting an injection tube into the fill material, such that an exit orifice of the injection tube is disposed in the fill material; and
driving the active pharmaceutical ingredient through the injection tube and out the exit orifice, such that the active pharmaceutical ingredient is delivered from the exit orifice and into the fill material.

2. The method of claim 1, wherein the inserting step comprises inserting the injection tube into the fill material along an insertion direction, and the method further comprises the steps of withdrawing the injection tube along a withdrawal direction opposite the insertion direction to a plurality of successive withdrawal positions, and the driving step is performed only when the injection tube is at the withdrawal positions.

3. The method of claim 1, wherein the delivered active pharmaceutical ingredient defines discrete zones disposed inside the outer wrapping, wherein the discrete zones are separate from each other and are adjoined to each other by a neck of delivered active pharmaceutical ingredient.

4. The method of claim 1, wherein the inserting step comprises inserting the injection tube into the fill material along an insertion direction, and the driving step is performed as the injection tube is withdrawn along a withdrawal direction opposite the insertion direction.

5. The method of claim 4, wherein the driving step is performed intermittently as the injection tube s withdrawn along the withdrawal direction.

6. The method of claim 4, wherein the active pharmaceutical ingredient is delivered continuously as the injection needle is withdrawn along the withdrawal direction.

7. The method of claim 1, wherein the exit orifice comprises a nozzle such that the active pharmaceutical ingredient is delivered from the nozzle along multiple directions that are angularly offset with respect to the central axis.

8. The method of claim 7, wherein at least 25% of the active pharmaceutical ingredient is delivered from the nozzle to a location that is beyond midway from the central axis to the outer wrapping.

9. The method of claim 7, wherein the active pharmaceutical ingredient is delivered radially from the nozzle.

10. The method of claim 9, wherein the nozzle is a spray nozzle, and the active pharmaceutical ingredient is delivered from the spray nozzle as an atomized spray.

11. The method of claim 7, wherein substantially all of the active pharmaceutical ingredient delivered from the exit orifice is delivered along the directions that are angularly offset with respect to the central axis.

12. The method of claim 1, wherein the pre-roll smokable structure comprises first and second open ends, and the inserting step comprises inserting the injection tube into the second open end toward the first open end, and the second end has a greater cross-sectional area than the first end.

13. The method of claim 1, wherein the inserting step comprises inserting the injection tube into the fill material substantially along the central axis, such that the active pharmaceutical ingredient is delivered from the exit orifice while the injection tube is disposed substantially along the central axis.

14. The method of claim 1, wherein the injection tube comprises a plurality of injection tubes that are spaced from each other.

15. The method of claim 14, wherein the inserting step comprises inserting the plurality of injection tubes parallel to each other.

16. The method of claim 14, wherein the exit orifice of each of the plurality of injection tubes are and remain aligned with each other along a plane as active pharmaceutical ingredient is delivered from the exit orifices, and the plane is oriented perpendicular to the central axis.

17. The method of claim 16, wherein the exit orifice of at least one of the injection tubes is inserted to a different depth with respect to the exit orifice of at least one other of the injection tubes.

18. The method of claim 1, wherein the active pharmaceutical ingredient comprises one or more of cannabidiol, tetrahydrocannabinol, and one or more terpenoids.

19. The method of claim 1, wherein the active pharmaceutical ingredient is substantially delivered substantially homogenously along the length of the pre-roll smokable structure.

20. The method of claim 1, wherein the pre-roll smokable structure comprises a tip end and a draw end, and a quantity of the active pharmaceutical ingredient increases from the tip end to the draw end.

22. The method of claim 1, wherein the pre-roll smokable structure comprises a tip end and a draw end, and a quantity of the active pharmaceutical ingredient increases from the draw end to the tip end.

23. The method of claim 1, wherein the pre-roll smokable structure is disposed in a chamber having a temperature less than an ambient temperature, and the active pharmaceutical ingredient is heated prior to travelling out of the exit orifice so as to decrease a viscosity of the active pharmaceutical ingredient, and the temperature quenches the active pharmaceutical ingredient after it has travelled out the exit orifice.

24. The method of claim 1, wherein microdroplets of the active pharmaceutical ingredient is delivered from the exit orifice and into the fill material.

25. The method of claim 1, further comprising the step of drying the fill material prior to surrounding the fill material with the outer wrapping.
